(19) United States
(12) Patent Application Publication
(10) Pub. No.: US 2010/0106136 A1
(43) Pub. Date: Apr. 29, 2010

(54) DRUG DELIVERY DEVICE WITH SLIDING CARTRIDGE

(75) Inventor: Thomas A. SIMONTON, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/260,673

(22) Filed: Oct. 29, 2008

(51) Int. Cl. A61M 5/315

(52) U.S. Cl. 604/511; 604/60

(57) ABSTRACT

Drug depot delivery devices and methods for delivering a drug depot to a site beneath the skin of a patient are provided. In various embodiments, a device for delivering a drug depot to a site beneath the skin of a patient is provided, the device comprising: a cannula capable of insertion to the site beneath the skin of the patient and having an opening for passage of the drug depot; a drug cartridge comprising at least two chambers, wherein each chamber is configured to store and release a drug depot into the cannula when the cannula is aligned with a first chamber and the plunger. In some embodiments, the drug depot delivery device and methods provide slide-a-dose technology for delivering the drug depot to the target tissue site.

### BACKGROUND

Drugs may be delivered to patients by a variety of methods including oral, intravenous, intramuscular, inhalation, topical, subcutaneous delivery or delivery directly or locally to the treatment site (e.g., intrathecally, intraspinally, intraarticularly, etc.). The method of delivery chosen depends, among other things, upon the condition being treated, desired therapeutic concentration of the drug to be achieved in the patient and the duration of drug concentration that must be maintained.

Recently, drug depots have been developed which allow a drug to be introduced or administered to sites beneath the skin of a patient so that the drug is slowly released over a long period of time. Such drug depots allow the drug to be released from the depot in a relatively uniform dose over weeks, months or even years. This method of administering drugs is becoming especially important and popular in modulating the immune, inflammation and/or pain responses in treatment of chronic conditions including rheumatoid arthritis, osteoarthritis, sciatica, carpal tunnel syndrome, lower back pain, lower extremity pain, upper extremity pain, cancer, tissue pain and pain associated with injury or repair of cervical, thoracic, and/or lumbar vertebrae or intervertebral discs, rotator cuff, articular joint, TMJ, tendons, ligaments, muscles, or a soft tissue surrounding the spinal canal, or the like.

Previously, drug depots and other types of implants have been inserted into the treatment site beneath the skin by use of a trocar device, which is a two-piece device that includes a cannula and an obdurator. The trocar device requires an incision to be made through the skin at the site of implant of the drug depot using a separate instrument (e.g., scalpel). A cannula and obdurator are inserted together through the skin at the incision site. Next, the obdurator is withdrawn, leaving the cannula in place as a guide for inserting the drug depot. The drug depot is inserted through the cannula, and the obdurator is used to push the implant to the end of the cannula. The cannula and obdurator are then withdrawn completely, leaving the implant in place beneath the skin.

Typically, trocar devices are used to implant drug depots subcutaneously over a large area (e.g., 2-2.5 inches), with a typical drug depot in the order of 1½ inches long. Thus, the trocar device is not suitable for many treatment sites because it lacks precision and may cause additional trauma to the tissue surrounding the site of implant.

Other drug depot devices have been developed to simplify implanting the drug depots. These devices have a handle for one-handed implantation of the drug depot, a needle containing the drug depot to be implanted and a rod positioned within the needle for pushing the drug depot out of the needle. Once the needle containing the drug depot has been inserted at the site of implant, a spring loaded trigger on the handle is activated which causes the needle to be automatically withdrawn by a spring leaving the implanted drug depot in place. Unfortunately, it is not possible to control the motion of the needle in these devices because the needle will automatically retract upon activation of the trigger. The complex spring loaded propelling system and trigger of these devices increase the chances that the device will jam and fail to eject the drug depot when required.

Conventional needle and syringe devices have been used to implant a drug depot to sites such as, for example, the epidural space. These devices typically utilize a syringe preloaded with the drug depot and an epidural needle. The needle is inserted through the skin, supraspinus ligament, intraspinus ligament, ligamentum flavum and then into the epidural space. The drug depot is delivered through the needle to the epidural space using the syringe plunger. Conventional needle and syringe devices often do not easily allow controlled and precision implant of the drug depot. If multiple drug depot implants are needed, these conventional needle and syringe devices often do not allow accurate placement of the implant in a manner so that one drug depot does not substantially interfere with the dissolution of the other.

New drug depot devices are needed, which can easily allow accurate and precise implantation of a drug depot with minimal physical and psychological trauma to a patient. When implanting several drug depots, a drug depot device is needed that accurately and precisely allows placement of the drug depot in a manner such that one depot does not substantially interfere with the dissolution of the others.

### SUMMARY

New drug depot devices, which can easily allow accurate and precise implantation of a drug depot with minimal physical and psychological trauma to a patient are provided. One advantage of the drug depot device is that it allows the user to dispense multiple doses of the drug in sequence.

The drug depot device, in various embodiments, includes a drug cartridge containing one or more drug pellets that has the advantages of easily being sterilized, the drug cartridge is coupled to a housing, a plunger in order to facilitate the release of the drug depot from the drug cartridge, and a cannula to deliver the drug depot to a site beneath the skin of a patient. In some embodiments, the drug depot device allows the user to “slide-a-dose” to deliver the drug depot. In some embodiments, the device contains a bulking agent and/or cover to hold the drug depot in position within the chambers, which makes for easier delivery of the drug depot. In some embodiments, the cover may be made of material that can be pierced by the plunger.

In one embodiment, a device is provided for delivering a drug depot to a site beneath the skin of a patient, the device comprising: a cannula having a proximal end and a distal end, the proximal end of the cannula having an opening to receive the drug depot, the distal end of the cannula capable of insertion to the site beneath the skin of the patient and having an opening for passage of the drug depot; a drug cartridge comprising at least two chambers, wherein each chamber is configured to store and release a drug depot into the cannula when the cannula is aligned with a first chamber; a housing having a top end, a bottom end, and a open portion, wherein the bottom end of the housing has a coupling means for coupling to the proximal end of the cannula and wherein the drug cartridge is slidably receivable within the open portion of the housing; and a plunger having a handle and a tip adapted for expelling a first drug depot stored in a first chamber, wherein the tip of the plunger is slidably receivable within each of the housing, the first chamber of the cartridge, and the cannula to deliver the first drug depot to the site beneath the skin of the patient when at least the first chamber is aligned with the cannula and the plunger.

In another embodiment, a kit is provided for delivering a drug pellet to a site beneath the skin of a patient, the kit comprising: a sterilized drug delivery device, comprising: a cannula having a proximal end and a distal end, the proximal end of the cannula having an opening to receive the drug pellet, the distal end of the cannula capable of insertion to the site beneath the skin of the patient and having an opening for passage of the drug pellet; a drug cartridge comprising at least two chambers, wherein each chamber is configured to store and release the drug pellet into the cannula when the cannula is aligned with a first chamber of the drug cartridge; a housing having a top end, a bottom end, and a open portion, wherein the bottom end of the housing has a coupling means for coupling to the proximal end of the cannula and the open portion is configured to receive the cartridge and wherein the drug cartridge is slidably receivable within the open portion of the housing; and a plunger having a handle and a tip adapted for expelling a first drug pellet stored in the first chamber, wherein the tip of the plunger is slidably receivable within each of the housing, the first chamber of the cartridge, and the cannula to deliver the first drug pellet to the site beneath the skin of the patient when at least the first chamber is aligned with the cannula and the plunger.

In yet another embodiment, a method is provided for delivering a drug pellet to a site beneath the skin of a patient, the method comprising: inserting a cannula at the target tissue site, the cannula having a proximal end and a distal end, the proximal end of the cannula having an opening to receive a drug pellet, the distal end of the cannula capable of insertion to the site beneath the skin of the patient and having an opening for passage of the drug pellet; attaching a drug cartridge to the proximal end of the cannula, the drug cartridge having a chamber containing the drug pellet; and sliding the cartridge to align the drug pellet with the proximal end of the cannula and a plunger, the plunger having a handle and a tip adapted for expelling the drug pellet from the cannula, wherein the tip of the plunger is slidably receivable within the chamber and the cannula to deliver the drug pellet to the site beneath the skin of the patient.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a side view of an embodiment of a drug depot delivery device having a cannula and a cartridge for storing a drug depot where the cannula and the cartridge are attached to a housing. In this embodiment, a plunger is inserted through the top of the housing, through the cartridge, and through the cannula for delivering the drug depot to a delivery site.

FIG. 1A illustrates a magnified cross-section view of the drug cartridge chamber filled with a bulking agent to fill the voids in the chamber and hold the drug pellet in position.

FIG. 2 illustrates a side view of an embodiment of the drug depot delivery device having a cannula, a cartridge for storing or holding a drug depot and a plunger for dispensing the drug depot from the cartridge to a drug depot delivery site. This embodiment illustrates the sliding cartridge and sliding cover that secures the drug depot inside the cartridge and an indicator means along the side of the cartridge, which serves to indicate alignment of the drug cartridge for dispensing the drug depot.

FIG. 3 illustrates a cross-sectional view of an embodiment of a drug depot delivery device having a cannula, a housing, a plunger, and a cartridge for storing a drug depot. In this illustrated embodiment, the drug chambers and drug pellets contained in the drug chambers are shown, as well as uncovered drug pellets ready for delivery.

FIG. 4 illustrates an embodiment of the assembly of a drug delivery device where the housing is being coupled to the proximal end of the cannula.

FIG. 5 illustrates an embodiment of the assembly of a drug delivery device where the cartridge is attached to the housing of the drug delivery device.

FIG. 6 illustrates an embodiment of the assembly of a drug delivery device where the plunger is inserted into the top of the housing to dispense the drug depot from the cartridge, through the cannula to a drug depot delivery site.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under any one heading may be used in conjunction with embodiments under any other heading.

New drug depot devices, which can easily allow the accurate and precise implantation of multiple drug depots with minimal physical and psychological trauma to a patient are provided. In various embodiments the drug depot device allows the user to dispense multiple drug depots, in sequence, to a site beneath the skin of the patient. In various embodiments, when several drug depots are to be implanted, a drug depot device is provided that accurately allows placement of the drug depot in a manner such that one depot does not substantially interfere with the dissolution of the others. In various embodiments, the drug depot device includes a drug cartridge containing one or more chambers for storing drug pellets, wherein the drug pellets can easily be sterilized and loaded into the drug depot device. In various embodiments, by using the drug cartridge having discrete regions to store delivery pellets, clustering of pellets and mis-delivery of drug pellets is reduced or avoided. In some embodiments, the drug depot device allows the user to “slide-a-dose” to deliver the drug dept. In some embodiments, the device contains a bulking agent and/or covers to hold the drug depot in position within the chambers, which makes for easier delivery of the drug depot.

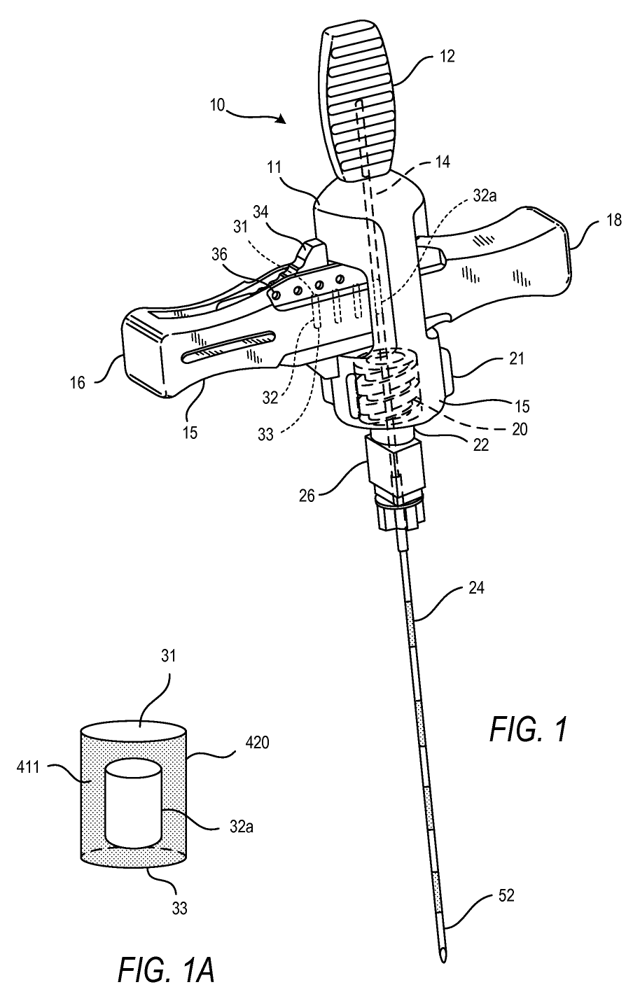

FIG. 1 illustrates various embodiments of an assembled drug delivery device 10 comprising a cannula 24, a drug cartridge 15 (proximal end shown as 16, and distal end shown as 18) and a plunger 14 all attached to housing 11. In various embodiments, the cannula 24 has a proximal end 22 and a distal end 52. The distal end of the cannula is capable of insertion to a site beneath the skin and the proximal end of the cannula 22 is capable of engaging a housing 11. In various embodiments, the proximal end of the cannula 22 is engaged to the housing with a coupling means 20, wherein the coupling means can be a luer lock, threading, friction fit fitting, etc. that engages distal end of the housing 15. In various embodiments, the cannula is hollow having a sufficient diameter to allow passage of a drug depot and the plunger 14 that facilitates delivery of the drug depot (e.g., drug pellet) to the designated site beneath the skin. The plunger 14 has handle 12 with grips for the user to move the plunger. The housing and cannula may also have grips 21 and 26 for the user to hold the housing and connect the cannula to it. The size of the cannula is dictated by the procedure to be performed.

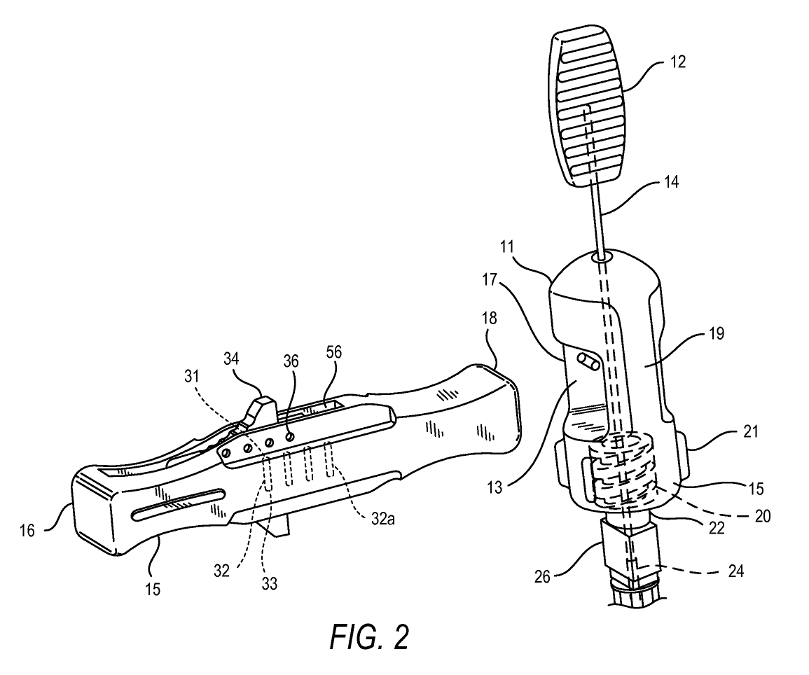

In various embodiments, a cartridge 15 is secured into the housing 11. In various embodiments, the cartridge 15 is perpendicular or substantially perpendicular to the axis of the housing or plunger and is adaptable for sliding in housing opening (13 in FIG. 2). In various embodiments, the housing has receiving columns, which may be contoured to allows the distal end 18 of the drug cartridge to slide within the receiving column to maintain the drug cartridge perpendicular or substantially perpendicular to the housing or cannula. Various embodiments provide an indicator means 36 to assist in aligning or indexing the drug cartridge 15 within the housing 11 so that the plunger 14, when moved in an extended position can dispense the drug pellet 32a from the proximal end 31 and distal end 33 of the drug chamber, through the cannula 24 to the drug delivery site.

In various embodiments, the plunger 14 is slideably engaged within the housing 11, and the cartridge 15 to move the drug pellet 32a in the chamber through the cannula 24 to deliver a drug pellet at the drug delivery site. In various embodiments, the plunger 14 is slideably inserted through an opening at the top of the housing 11, through the drug cartridge 15, dislodging a drug pellet 32a loaded in the drug chamber, pushing the drug pellet through the cannula 24 and delivering the drug pellet through the distal end of the cannula 52 to the delivery site. The plunger 14 may have a knob or handle 12 on one end to assist the user in manipulating the plunger 14, enabling the user to direct the drug pellet to the delivery site. The knob or handle 12 may be provided in various shapes in relative proportion to the size of the assembled delivery device.

In some embodiments, the drug cartridge 15 may have a cover 34 disposed on upper portion 31 of the drug chamber and the lower portion of the drug chamber 33, while the drug pellet 32 is disposed therein. The cover contains the drug pellet within the chamber. When the chamber is uncovered, the pellet is uncovered (shown in 32a) and is ready for delivery. Like the drug cartridge 15, the cover 34 may be substantially perpendicular to the housing and/or plunger and is slidably disposed on the drug cartridge. The user can slide the cover in the same or opposite direction of the cartridge to uncover the drug chamber and thus uncover the drug pellet 32a so that the plunger now can push the drug pellet out of the cartridge 15 and deliver the drug pellet 32a to the cannula 24.

### Cannula or Needle

The cannula or needle of the drug depot device is designed to cause minimal physical and psychological trauma to the patient. Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, nitinol, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The cannula or needle of the drug depot device has a diameter that is larger than the diameter of at least part of the plunger (e.g., tip, middle, etc.) to allow at least part of the plunger to be slidably received within the cannula or needle. In various embodiments, the diameter of the cannula or needle is substantially the same throughout. In other embodiments, the diameter of the needle or cannula becomes smaller approaching the distal end for drug delivery.

The dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. Some examples of lengths of the cannula or needle may include, but are not limited to, from about 50 to 150 mm in length, for example, about 65 mm for epidural pediatric use, about 85 mm for a standard adult and about 150 mm for an obese adult patient. The thickness of the cannula or needle will also depend on the site of implantation. In various embodiments, the thickness includes, but is not limited to, from about 0.05 to about 1.655. The gauge of the cannula or needle may be the widest or smallest diameter or a diameter in between for insertion into a human or animal body. The widest diameter is typically about 14 gauge, while the smallest diameter is about 25 gauge. In various embodiments the gauge of the needle or cannula is about 17 to about 25 gauge.

In various embodiments, the plunger, cannula or drug depot include markings that indicate location at or near the site beneath the skin. Radiographic markers can be included on the drug depot to permit the user to accurately position the depot into the site of the patient. These radiographic markers will also permit the user to track movement and degradation of the depot at the site over time. In this embodiment, the user may accurately position the depot in the site using any of the numerous diagnostic-imaging procedures. Such diagnostic imaging procedures include, for example, X-ray imaging or fluoroscopy. Examples of such radiographic markers include, but are not limited to, barium, calcium phosphate, and/or metal beads.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography.

In various embodiments, the drug depot comprises a drug cartridge containing drug pellets loaded within the chamber of the drug cartridge, when the plunger is moved to the extended position, the drug cartridge will remain within the housing and the chamber of the drug cartridge will guide the tip of the plunger longitudinally and the drug pellet will be released from it when it is in the extended position. A subsequent or second pellet may be administered by repositioning the needle at a target site, removing the plunger so that it is at a position above the drug cartridge, and rotating the drug cartridge at a position horizontal to the plunger and cannula to align the drug chamber and drug depot with the cannula and plunger. The plunger is then slid in a vertical direction within the housing to release the drug depot from the chamber into the cannula where the drug depot can be delivered to the target site by pushing it out the tip of the needle using the plunger. In this way, sequential delivery of a drug can be accomplished. Thus, the above procedure (e.g., repositioning the needle, removing the plunger, rotating the drug cartridge, inserting the plunger, delivering the drug depot) can be repeated multiple times to deliver multiple drug depots to the target tissue site.

In various embodiments, surrounding the opening of the proximal end of the cannula or needle is a generally cylindrical hub having an engagement means (shown as internal threading) for engaging the housing. Engagement means include, but are not limited to, threading, tracks, clips, ribs, projections, and the like that allow a secure connection between the housing and the proximal end of the cannula. For example, in various embodiments the engagement means may be a luer lock connection, where the cannula has mating threads that mate with the threads disposed on or in the housing.

### Housing

The housing may be of various shapes including, but not limited to, square, rectangular, cylindrical, round such that the housing allows for the affixation to the cannula as well as the drug cartridge and the plunger.

The housing may comprise a variety of materials, such as, for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof.

Like the cannula or needle, in various embodiments, the housing may have dose indicator markings (e.g., numbers, lines, letters, radiographic markers, etc.) to indicate the number of drug depots delivered. In various embodiments, the plunger includes markings that indicate location at or near the site beneath the skin.

The housing may have contours and allow easy grasping of the device during use for insertion of the drug depot. The housing can be angled for right and left hand users or can be generic for both hands. In various embodiments, the housing can comprise an upper opening, a middle opening, and a lower opening. The upper, middle and lower openings allow a plunger to slide through the openings. The middle opening of the housing, in various embodiments, will receive the drug cartridge and the user can align the chamber of the drug cartridge with the upper middle and lower openings so that the plunger can pass through and deliver the drug depot.

### Plunger

Although the first end of the plunger is shown as a knob, it will be understood that the knob can be a top, cap, handle or any member that allows the user to utilize the plunger. The plunger has a second end that includes a tip, which is capable of moving the drug depot within the cannula. In other embodiments, the tip of the plunger is sufficiently pointed so that it is capable of insertion to the site beneath the skin of the patient and the cannula or needle is blunted and used to guide the drug depot to the site.

The plunger has a diameter less than the cannula or needle so that it can be slidably received therein. The plunger may be longer, the same size, or smaller in length than the cannula or needle. In embodiments where the plunger extends from the distal end of the cannula or needle, the plunger is usually longer than the cannula or needle. In some embodiments, the tip of the plunger can be sharp or blunt. The sharper tip of the plunger can be used in embodiments where the drug cartridge has superior and inferior covers that the sharp tip of the plunger can pierce.

The plunger may be made from materials, such as for example, polyurethane, polyurea, polyether(amide), PEBA, thermoplastic elastomeric olefin, copolyester, and styrenic thermoplastic elastomer, steel, aluminum, stainless steel, titanium, nitinol, metal alloys with high non-ferrous metal content and a low relative proportion of iron, carbon fiber, glass fiber, plastics, ceramics or combinations thereof. The plunger may optionally include one or more tapered regions.

Like the cannula or needle, in various embodiments, the plunger may have dose indicator markings (e.g., numbers, lines, letters, radiographic markers, etc.) to indicate the number of drug depots delivered. In various embodiments, the plunger includes markings that indicate location at or near the site beneath the skin.

The plunger tip, which may be a complementary shape to the drug pellet, allows the plunger tip to snuggly fit within the end of the drug pellet for easier drug delivery. The drug pellet may have a rounded end for easier insertion at the desired site.

### Drug Depot

In various embodiments, the device comprises a drug depot. A drug depot comprises a physical structure to facilitate implantation and retention in a desired site (e.g., a synovial joint, a disc space, a spinal canal, a tissue of the patient, etc.). The drug depot also comprises the drug. The term “drug” as used herein is generally meant to refer to any substance that alters the physiology of the patient. The term “drug” may be used interchangeably herein with the terms “therapeutic agent”, “therapeutically effective amount”, and “active pharmaceutical ingredient”. It will be understood that a “drug” formulation may include more than one therapeutic agent, wherein exemplary combinations of therapeutic agents include a combination of two or more drugs. The drug provides a concentration gradient of the therapeutic agent for delivery to the site. In various embodiments, the drug depot provides an optimal drug concentration gradient of the therapeutic agent at a distance of up to about 1 mm to about 5 cm from the implant site.

Examples of drugs suitable for use in the drug depot, include, but are not limited to an anti-inflammatory agent, analgesic agent, or osteoinductive growth factor or a combination thereof. Anti-inflammatory agents include, but are not limited to, salicylates, diflunisal, indomethacin, ibuprofen, naproxen, tolmetin, ketorolac, diclofenac, ketoprofen, fenamates (mefenamic acid, meclofenamic acid), enolic acids (piroxicam, meloxicam), nabumetone, celecoxib, etodolac, nimesulide, apazone, gold, sulindac or tepoxalin; antioxidants, such as dithiocarbamate, and other compounds such as sulfasalazine [2-hydroxy-5-[-4-[C2-pyridinylamino)sulfonyl]azo]benzoic acid], steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof, or protein inhibitors of TNF, such as etanercept, Remicade, IL-1, such as Kineret®, p38, RANK, RANKL or a combination thereof.

Suitable osteoinductive factors include, but are not limited to, a bone morphogenetic protein, a growth differentiation factor, a LIM mineralization protein or a combination thereof.

Suitable analgesic agents include, but are not limited to, acetaminophen, lidocaine, bupivicaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine or a combination thereof. Analgesics also include agents with analgesic properties, such as for example, amitriptyline, carbamazepine, gabapentin, pregabalin, clonidine, or a combination thereof.

A “depot” includes but is not limited to capsules, microspheres, particles, coating, matrices, wafers, pills, pellets or other pharmaceutical delivery compositions. In various embodiments, the depot may comprise a bioerodible, a bioabsorbable, and/or a biodegradable biopolymer that may provide immediate release, or sustained release of the drug. Examples of suitable sustained release biopolymers include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, ,-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. As persons of ordinary skill are aware, mPEG may be used as a plasticizer for PLGA, but other polymers/excipients may be used to achieve the same effect. mPEG imparts malleability to the resulting formulations. In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

In various embodiments, the drug depot comprises drug pellets loaded with a therapeutically effective amount of the therapeutic agent, wherein the pellets are injected into a synovial joint, a disc space, a spinal canal, or a soft tissue surrounding the spinal canal. In various embodiments, the drug pellets comprise a gel in viscous form and microspheres loaded with a therapeutic agent, wherein the combination of gel and microspheres are positioned into a synovial joint, disc space, a spinal canal, or a soft tissue surrounding the spinal canal of a subject.

A “therapeutically effective amount” is such that when administered, the drug results in alteration of the biological activity, such as, for example, inhibition of inflammation, reduction or alleviation of pain, improvement in the condition, etc. The dosage administered to a patient can be as single or multiple doses depending upon a variety of factors, including the drug's pharmacokinetic properties, the route of administration, patient conditions and characteristics (sex, age, body weight, health, size, etc.), extent of symptoms, concurrent treatments, frequency of treatment and the effect desired.

In one exemplary embodiment, the drug depot is in the form of a pellet. The pellet can be any shape, such as for example, bullet shaped, spherical, substantially spherical, flaked, rod shaped, square, oval, etc. In various embodiments, the pellet has an aspect ratio (a ratio of the length of the pellet divided by the width found at an angle of 90° in respect to the length), which is less than about 1.4 to about 1.05. The proximal end of the drug pellet may allow the plunger tip to snuggly fit within the proximal end of the drug pellet for easier drug delivery. The distal end of the drug pellet may be rounded for easier insertion at the site.

In various embodiments, the drug pellet comprises a bullet-shaped body that is made from a biodegradable material. In alternative embodiments, the body of the pellet may be made from a non-biodegradable material. A non-biodegradable body could be a porous hollow chamber filled with the therapeutic agent alone or incorporated into a degradable polymer. It may be desirable to make the body non-degradable to be able to retrieve it after it has released its contents. Non-limiting examples of suitable biodegradable materials for the pellet body include polyorthoesters (POE), polylacticglycolic acid (PLGA) polysacharides (Saber technology), polycapralactone, polyfumarate, tyrosine polycarbonate, etc. The body may be solid, and the therapeutic agent may be dispersed throughout the material that forms the body. The dispersal of the therapeutic agent may be even throughout the body. Alternatively, the concentration of the therapeutic agent may vary throughout the body. As the biodegradable material of the body degrades at the site, the therapeutic agent is released.

Procedures for making pellets include, but are not limited to, extrusion-spheroidization, for spherical pellets where the active pharmaceutical ingredient (API) and any inactive ingredients (excipients, binders, etc.) are pre-mixed, then wetted with water, in a high shear mixer to form a damp mass. The damp mass is then transferred into an extruder where it is forced through a screen or die plate, where it forms an essentially solid, cylindrical extrudate of uniform shape and size. The size of the opening in the screen or die dictate resultant pellet size. The extrudate is fed onto a rotating disk, which may be smooth or may contain a grid (waffled, grooved, etc.) and the extrudate breaks into small cylinders, which in time are rounded into spherically shaped solids. Subsequently, the pellets are dried to the desired residual moisture content, typically in a fluid bed dryer. Any oversized or undersized product is removed by sieving, and the resulting pellets have a narrow size distribution.

In various embodiments, the API is layered on the solid core of the pellet by solution or suspension layering or powder layering techniques. In solution or suspension layering, an API and any inactive ingredients (excipients, binders, etc.) are suspended or dissolved in water or an organic solvent. The resulting liquid is sprayed onto the outside of a core particle, which may include, for example, non-pareil sugar seed (sugar sphere), microcrystalline cellulose pellets and the like, to make the pellet having the desired potency. Solution or suspension layering may be conducted using a wide variety of process techniques, for example, by fluidized bed, Wurster bottom spray techniques, or the like. When the desired potency has been achieved, pellets are dried to the desired residual moisture content. Any oversized or undersized product may be removed by sieving, and the resulting pellets are narrow in size distribution.

Powder layering may also be used to make the drug pellets. Powdered layering involves the application of a dry powder to the pellet core material. The powder may contain the drug, or may include excipients such as a binder, flow aid, inert filler, and the like. In the powder layering technique a pharmaceutically acceptable liquid, which may be water, organic solvent, with or without a binder and/or excipients, is applied to the core material while applying the dry powder until the desired potency is achieved. When the desired potency has been achieved, the pellets may be seal coated to improve their strength, and are then dried to the desired moisture content. Any oversized or undersized product is removed by sieving, and the resulting pellets are narrow in size distribution.

In one embodiment, the pellet is made using a core of biodegradable material, such as, for example, polyglactin, polylactone, polylactide, etc. The core is then coated with a thin layer of the API, such as an anti-inflammatory agent, analgesic agent, etc. by solution, suspension, or powdered layering until the desired potency is achieved.

In various embodiments, the drug pellets can be different sizes, for example, from about 1 mm to 5 mm in length and have a diameter of from about 0.01 to about 2 mm, or 0.5 mm and 0.8 mm. The layer or layers will each have a layer thickness of from about 0.005 to 1.0 mm, such as, for example, from 0.05 to 0.75 mm. The drug depot chambers are often larger than the drug depot dimensions to keep the drug depot within the drug chamber.

Like the cannula, needle, or plunger, in various embodiments, the drug depot (e.g., pellet, cartridge, etc.) may have dose indicator markings (e.g., numbers, lines, letters, radiographic markers, etc.) to indicate the number of drug depots delivered. In various embodiments, radiopaque marks are positioned on the depot at opposite ends of the depot to assist in determining the position of the depot relative to the treatment site. For example, the radiopaque marker could be a spherical shape or a ring around the depot.

### Drug Cartridge

In various embodiments, the drug depot is stored in a drug cartridge. The drug cartridge comprises one or more chambers, each chamber capable of storing a drug pellet. Each chamber isolates the drug pellet from contact with other drug pellets contained within the cartridge. In this way, overcrowding or multiple pellets in one chamber of the drug cartridge is avoided. Further, drug pellets falling out of the drug cartridge due to limited space in the cartridge is also avoided.

In various embodiments, the drug cartridge is circular having an opening for attaching to the housing structure of the drug depot delivery device. For example, the drug cartridge can be affixed to a column of the housing and the user slides the cartridge in the housing to align the pellet with the plunger and cannula for delivery. In various embodiments, the drug cartridge is linear and is slidably receivable through the opening of the housing such that the cartridge is perpendicular to the housing. For example, the drug cartridge may be a rectangular shape and slide within the opening of the housing at a position perpendicular to the plunger. To deliver the drug depot, the cartridge slides with the housing to align with cannula and plunger. The plunger then slides through the cannula to deliver the drug depot through the cannula and out to the target site. In various embodiments, the cartridge comprises superior and inferior covers to contain the drug pellet in the chambers to avoid slippage of the pellets from the cartridge.

In various embodiments, the drug cartridge may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or a combination thereof. In various embodiments, the drug cartridge is not biodegradable.

In some embodiments, the drug cartridge comprises 1, 2, 3, 4, 5, 6, 7, 8, 9, 10, 11, 12, 13, 14, 15, 16, 17, 18, 19, and 20 drug chambers, where each chamber comprises one drug depot. The chambers can be spaced an equal distance from each other. For example, the drug chamber can be spaced 0.5 mm, or 1 mm, or 5 mm, or 1 cm to about 2 cm from each other.

In various embodiments, the drug depot is secured within a chamber by a cover to keep the drug depot in place preventing the drug depot from slipping from the cartridge. In various embodiments, the cover may be made from the same material used to make the drug cartridge, or any other exemplary material that could be used to make the drug cartridge.

In the embodiments of the cartridge where the covers are not penetrable, when the cartridge is affixed to the body of the housing, only the chambers not aligned for delivery of the drug depot are covered by the cover. For example, when the cover is not penetrable by the plunger, it is substantially contiguous with the drug cartridge such that the surface of the cover contacts the drug cartridge, except for the region of the drug cartridge that contacts the housing as this region will be aligned with the plunger and cannula for delivery of the drug depot (an uncovered pellet is shown in FIG. 1 as 32a and in FIG. 1A as 32a).

In various embodiments, the cover securing the drug depot in the chamber in the drug cartridge is made of a thin layer of material that can be penetrated and can be cored by the plunger and/or depot in order to release the drug depot. In various embodiments the penetrable material may comprise, for example, a bioerodible, a bioabsorbable, and/or a biodegradable biopolymer. Examples of suitable materials include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), mPEG, poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, ε-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), wax, agar, agarose, gel-vitamin or combinations thereof. In various embodiments, the cover comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

### Bulking Agent

In various embodiments, the drug depots are secured in the drug cartridge by use of a bulking agent. The bulking agent may be added to the drug depot to ensure the drug depot is secure within the chamber, such that the drug depot is released when the plunger is engaged to dislodge the drug depot from the cartridge. The bulking agent can be added before, during or after the drug depot is placed in the chamber of the drug cartridge. In some embodiments, a method is provided of partially filling the chamber with the bulking agent and then placing the drug depot in the chamber. In some embodiments, a method is provided of placing the drug depot within the chamber and then partially or completely filling the chamber with the bulking agent.

In some embodiments, the bulking agent can be penetrated and can be cored by the plunger and/or depot in order to release the drug depot. A bulking agent includes an excipient, which provides bulk and structure to the drug depot and holds the drug depot in position within the chamber. In some embodiments, the bulking agent prevents unwanted movement, contaminants (e.g., moisture), and breakage of the drug depot. In some embodiments, the bulking agent fills the space within the chamber so that there is little or no repositioning of the drug depot during delivery. Examples of suitable bulking agents include hydrophilic excipients, such as, water soluble polymers; sugars, such as mannitol, sorbitol, xylitol, glucitol, ducitol, inositiol, arabinitol, arabitol, galactitol, iditol, allitol, maltitol, fructose, sorbose, glucose, xylose, trehalose, allose, dextrose, altrose, lactose, talc, zinc oxide, starch, hydroxyethylstarch (hetastarch), cellulose, cyclodextrins, glycine, fructose, gulose, idose, galactose, talose, ribose, arabinose, raffinose, xylose, lyxose, sucrose, maltose, lactose, lactulose, fucose, rhamnose, melezitose, maltotriose, raffinose, altritol, their optically active forms (D- or L-forms) as well as the corresponding racemates; inorganic salts, both mineral and/or mineral organic, such as, calcium salts, such as the lactate, gluconate, glycerylphosphate, citrate, phosphate monobasic and dibasic, succinate, sulfate and tartrate, as well as the same salts of aluminum and magnesium; carbohydrates, such as, the conventional mono- and di-saccharides as well as the corresponding polyhydric alcohols; proteins, such as, albumin; amino acids, such as glycine; emulsifiable fats or polyvinylpyrrolidone or a combination thereof. Exemplary bulking agents include glycine, mannitol, dextran, dextrose, lactose, sucrose, polyvinylpyrrolidone, trehalose, glucose, wax, agar, agarose, gel-vitamin or combinations thereof. The bulking agent may be in solid, semisolid, or liquid form. In various embodiments, the bulking agent is in a powdered form.

In some embodiments, the particle size of the solid or semi-solid bulking agents range is greater than 10 microns as particles of this size are easily removed by macrophages and other cells of the immune system. In some embodiments, the particle size of the solid or semi-solid bulking agents range from about 10 microns to about 1500 microns in diameter, or from about 150 microns to about 1100 microns in diameter, or from about 500 microns to about 900 microns in diameter. The size of the particles chosen for a particular application will be determined by a number of factors. Smaller particles are easier to inject with a smaller gauge size needle. The size of the particles used in a particular procedure will include consideration of the procedure employed, disease progression, the degree of degradation of the affected region, patient size, the disposition of the patient, and the preferences and techniques of the doctor performing the procedure.

In some embodiments, the bulking agent does not contain a therapeutic agent. In other embodiments, a therapeutic agent may be dispersed throughout the bulking agent and provide immediate release of the therapeutic agent.

In some embodiments, the bulking agent includes a bioerodible, a bioabsorbable, and/or a biodegradable biopolymer like the depot and/or superior and/or inferior covers. Examples of suitable materials include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), mPEG, poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, ε-caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) or combinations thereof. In various embodiments, the bulking agent comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone or a combination thereof.

The drug device components (e.g., cannula or needle, plunger, housing, engagement means, etc.) may be lightweight, disposable and sterilizable such that when the device is assembled (e.g., the drug cartridge is attached to the housing), the weight of the device does not substantially increase. In various embodiments, one or more components of the device are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, the drug cartridge provides the advantages of ease of manufacturing in the terminal sterilization process. If the drug pellets are preloaded in the manufacturing process, gamma radiation may be required at higher doses to sterilize the drug depot loaded in the cannula or needle. This is particularly so when the cannula or needle is made from steel or metal. Thus, to sterilize the loaded depot, the dose of gamma rays must be high enough to penetrate the metal, which may destroy the API in the drug depot. By providing a drug cartridge, for example, made of plastic, the drug cartridge and drug pellets in the cartridge can be sterilized, without destroying the API and then subsequently loaded by the manufacturer or the user (e.g., surgeon, physician, nurse, etc.). Further, loading the drug depot into the drug chamber or cannula is easier. This is particularly so when dealing with multi-dose drug pellets that are relatively small (e.g., 1 mm to 5 mm), the user typically cannot grasp these small pellets and load them into the device. By providing them in a drug cartridge, the user does not have to substantially manipulate the individual drug pellets and the risk of contaminating the pellets particularly with sterilized pellets is reduced.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot includes a gelatin capsule.

Other methods may also be used to sterilize one or more components of the device, including, but not limited to, gas sterilization, such as, for example, with ethylene oxide or steam sterilization.

In some embodiments, the housing, drug cartridge, and/or cannula are transparent so the user can see the position of the plunger and/or the drug depot in the chamber of the drug cartridge. Thus, indicator markings, in this embodiment, are not needed.

In various embodiments, a kit is provided which may include additional parts along with the drug depot device combined together to be used to implant the drug depot. The kit may include the drug depot device in a first compartment. The second compartment may include the drug cartridge, and any other instruments needed for the implant. A third compartment may include gloves, drapes, wound dressings and other procedural supplies for maintaining sterility of the implanting process, as well as an instruction booklet. A fourth compartment may include additional cannulas and/or needles. Each tool may be separately packaged in a plastic pouch that is radiation sterilized. A cover of the kit may include illustrations of the implanting procedure and a clear plastic cover may be placed over the compartments to maintain sterility.

In various embodiments, a method is provided for delivering a drug depot to a site beneath the skin of a patient, the method comprising: assembling a drug delivery device wherein the drug delivery device comprises a cannula having a proximal end and a distal end, the proximal end of the cannula having an opening to receive the drug depot, the distal end of the cannula capable of insertion to the site beneath the skin of the patient and having an opening for passage of the drug depot; a cartridge comprising at least a first secure chamber and a second secure chamber, wherein each chamber is capable of storing one drug pellet; a housing having a top end, a bottom end, and an open portion, wherein the bottom end of the housing has a coupling means for coupling to the proximal end of the cannula and wherein the open center is adaptable to receive the cartridge; a plunger having a knob end and a tip end adaptable for expelling the drug pellet, from each chamber, wherein the tip end is slidably receivable within each of the housing, the cartridge, and the cannula to deliver the drug pellet to the site beneath the skin of the patient; selecting a drug delivery site beneath the skin of the patient; and dispensing the drug pellet from the drug delivery device to a site beneath the skin of the patient.

In various embodiments, the seal between the plunger tip and the cannula or needle can be air tight so that when the cannula or plunger penetrates the skin, at times, fluid (e.g., blood, spinal fluid, synovial fluid, etc.) may be drawn up into the cannula or needle. This fluid will be expelled when the plunger is re-inserted into the cannula or needle and the drug depot is released.

The device may be used for localized and/or targeted delivery of the drug to a patient to treat a disease or condition such as for example, rheumatoid arthritis, osteoarthritis, sciatica, carpal tunnel syndrome, lower back pain, lower extremity pain, upper extremity pain, cancer, tissue pain and pain associated with injury or repair of cervical, thoracic, and/or lumbar vertebrae or intervertebral discs, rotator cuff, articular joint, TMJ, tendons, ligaments, bone muscles, or soft tissue surrounding the spinal canal, or the like.

In various embodiments, the drug depot device is used to treat pain, or other diseases or conditions of the patient. Pain includes acute pain and neuropathic pain. Acute pain refers to pain experienced when tissue is being damaged or is damaged (e.g., injury, infection, etc.). As contrasted to acute pain, neuropathic pain serves no beneficial purpose. Neuropathic pain results when pain associated with an injury or infection continues in an area once the injury or infection has resolved. Sciatica provides an example of pain that can transition from acute to neuropathic pain. Sciatica refers to pain associated with the sciatic nerve which runs from the lower part of the spinal cord (the lumbar region), down the back of the leg and to the foot. Sciatica generally begins with a herniated disc. The herniated disc itself leads to local immune system activation. The herniated disc also may damage the nerve root by pinching or compressing it, leading to additional immune system activation in the area.

Patients include a biological system to which a treatment can be administered. A biological system can include, for example, an individual cell, a set of cells (e.g., a cell culture), an organ, or a tissue. Additionally, the term “patient” can refer to animals, including, without limitation, humans.

Treating or treatment of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols that have only a marginal effect on the patient.

“Localized” delivery includes, delivery where one or more drugs are deposited within a tissue, for example, a nerve root of the nervous system or a region of the brain, or in close proximity (within about 10 cm, or preferably within about 5 cm, for example) thereto. “Targeted delivery system” provides delivery of one or more drugs depots in a quantity of pharmaceutical composition that can be deposited at the target site as needed for treatment of pain, inflammation or other disease or condition.

FIG. 1A illustrates a magnified view of an uncovered drug pellet 32a that is loaded in drug chamber 420 substantially perpendicular to housing of the drug cartridge. In this way, the drug pellets are in an upright position within the chamber. Upper opening 31 of the chamber is of a diameter to receive a plunger and drug pellet and lower opening 33 is of a diameter to allow exit of the drug pellet and plunger. In some embodiments, the drug pellet is snuggly fit within the chamber so that some force may be required by the plunger to push the drug pellet out of the chamber. In some embodiments, optionally there is a bulking agent shown as 411 that surrounds at least a portion or all of the drug pellet. In some embodiments, the bulking agent holds the drug pellet in position and prevents unwanted movement, contaminants (e.g., moisture), and breakage of the drug pellet. The bulking agent fills the space within the chamber so that there is little or no repositioning of the pellet during drug delivery. It will be understood by those of ordinary skill in the art that the bulking agent can be disposed in discrete regions of the drug chamber and not in others. For example, the bulking agent can be disposed only around the drug depot in the upper part of the chamber near 31 or lower part of the chamber near 33 to prevent unwanted movement of the drug pellet 32 a before or during drug delivery. In FIG. 1A, the drug chamber 420 and thus the drug pellet 32a is uncovered.

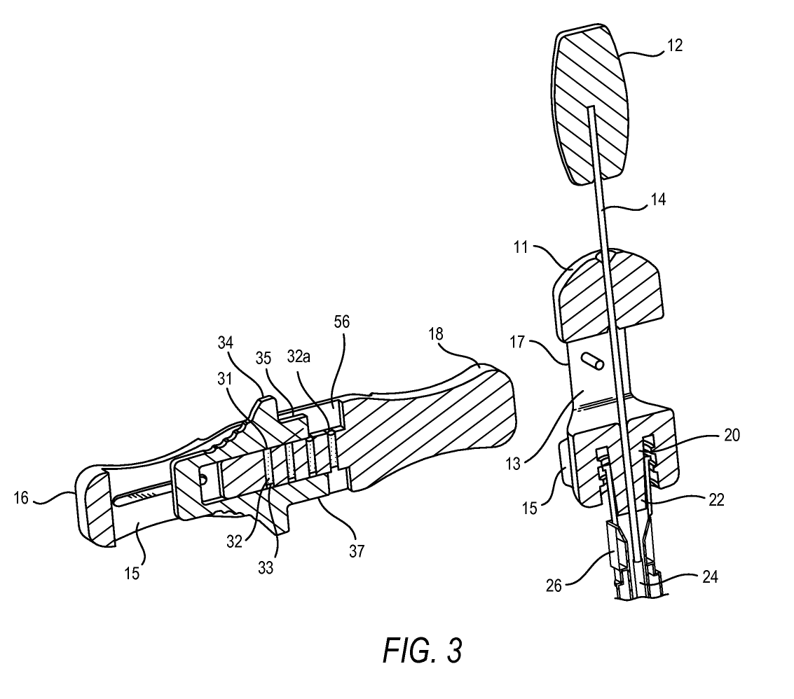

FIGS. 2-3 illustrate a side view of the partially assembled drug delivery device comprising a portion of the cannula 24, a drug cartridge 15 (proximal end shown as 16 and distal end shown as 18) separated from the housing 11. The plunger 14 shown in the partially extended position is attached to housing 11. In various embodiments, the cannula 24 has a proximal end 22 and a distal end opening (not shown). The distal end of the cannula is capable of insertion to a site beneath the skin and the proximal end of the cannula 22 is capable of engaging a housing 11. In various embodiments, the proximal end of the cannula 22 is engaged to the housing with a coupling means 20, wherein the coupling means can be a luer lock, threading, friction fit fitting, etc. that engages distal end of the housing 15. In various embodiments, the cannula is hollow having a sufficient diameter to allow passage of a drug depot and the plunger 14 that facilitates delivery of the drug to the designate site beneath the skin. The plunger 14 has handle 12 with grips for the user to move the plunger. The housing and cannula may also have grips 21 and 26 for the user to hold the housing and connect the cannula to it. The size of the cannula is dictated by the procedure.

In various embodiments, a cartridge 15 is secured to the housing 11 at opening 13, where the cartridge is configured to slide between the columns of the housing 17 and 19 through opening 13. The columns act as a channel to guide the distal end of the cartridge 18 through opening 13. In this way the drug chamber containing drug pellet 32a can be aligned with the cartridge opening 56, the plunger 14 and cannula 24 so that the plunger can pass the drug pellet through the cannula and out to the target tissue site. In some embodiments, the housing will have one or more projections (shown near 17) that may aid in guiding the drug cartridge through the housing.

In various embodiments, the cartridge 15 is perpendicular or substantially perpendicular to the axis of the housing or plunger and is adaptable for attaching to the housing at an opening 13 disposed within the columns 17 and 19 of the housing. In various embodiments, the housing has receiving columns (17 and 19), which may be contoured to allow the distal end 18 of the drug cartridge to slide within the receiving columns of the housing to maintain the drug cartridge perpendicular or substantially perpendicular to the housing or cannula. Various embodiments provide an indicator means 36 to assist in aligning the drug cartridge 15 and opening 56 that is parallel to the drug chamber containing the drug pellet 32a. Once aligned, the housing 11, chamber, drug pellet 32a, and cannula 24 will have aligned holes that will allow the plunger 14 to pass through the housing 11, chamber, and cannula 24 and move in an extended position the drug pellet 32a from the drug chamber, through the cannula 24 to the drug delivery site. In various embodiments, the device will be single dose. In other embodiments, the device is multi-dose and contains two or more pellets (32 and 32a), which when aligned, allows the plunger to enter proximal opening 31 of the drug chamber and push the drug pellet out distal opening 33.

In various embodiments, the plunger 14 is slideably engaged within the housing 11, and the cartridge 15 to move the drug pellet 32a in the chamber through the cannula 24 to deliver a drug pellet at the drug delivery site. In this way, the user can “slide-a-dose” using the device. In various embodiments, the plunger 14 is slideably inserted through an opening at the top of the housing 11, through the drug cartridge 15, dislodging a drug pellet 32a loaded in the drug chamber, pushing the drug pellet through the cannula 24 and delivering the drug pellet 32a through the distal end of the cannula to the delivery site. The plunger 14 may have a knob 12 on one end to assist the user in manipulating the plunger 14, enabling the user to direct the drug pellet to the delivery site. The knob or handle 12 may be provided in various shapes in relative proportion to the size of the assembled delivery device.

In some embodiments, the drug cartridge 15 may have a cover 34 disposed on upper portion 31 of the drug chamber and the lower portion of the drug chamber 33, while the drug pellet 32 is disposed therein. The cover contains the drug pellet within the chamber when it covers the chamber. When the cover is moved to uncover the pellet (shown in 32a), the pellet is ready for delivery from the chamber. Like the drug cartridge 15, the cover 34 may be substantially perpendicular to the housing and/or plunger and is slidably disposed on the drug cartridge. The user can slide the cover in the same or opposite direction of the cartridge to uncover the drug chamber and thus uncover the drug pellet 32a so that the plunger now can push the drug pellet out of the cartridge 15 and deliver the drug pellet 32a to the cannula 24.

In various embodiments, the cartridge 15 is rectangular shape as shown in FIG. 2. In various embodiments, the cover snap-fits within the drug cartridge through opening 56 and the user can align the drug pellet for delivery by moving the cover 34 forward or backward or move the housing 15 forward or backward when the cartridge is disposed in the housing at opening 13 between columns 17 and 19.

FIG. 3 illustrates a cross-sectional view of an embodiment of a drug depot delivery device having a cannula, a housing, a plunger, and a cartridge for storing a drug depot. In this illustrated embodiment, the drug chambers and drug pellets contained in the drug chambers are shown, as well as uncovered drug pellets ready for delivery. In FIG. 3, the upper portion of the cover 35 and lower portion of the cover 37, both cover upper opening 31 and lower opening 33 of the drug chamber. Drug pellet 32a is shown in an uncovered position and allows the plunger 14 to push the pellet out of the cannula 24 to the target delivery site.

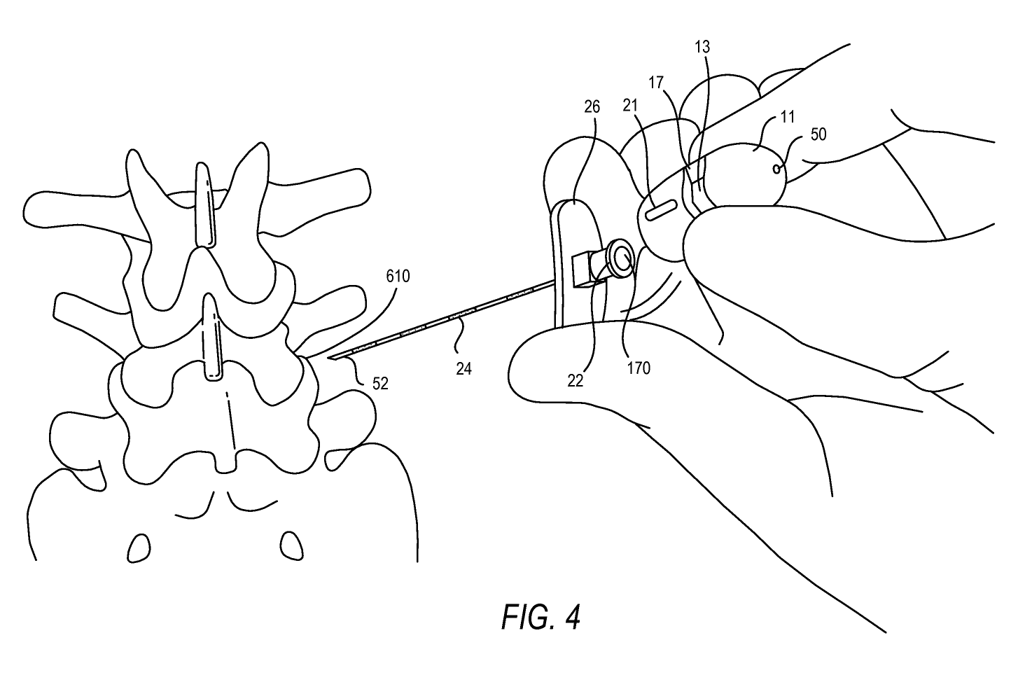

FIGS. 4-6 illustrate exemplary embodiments of application of a drug delivery device being assembled and having a cannula 24 coupled, by a coupling means 170 at the proximal end of the cannula 22 to a housing 11. In the exemplary embodiments shown, the distal end of the cannula 52 is positioned to deliver the drug depot to a delivery site beneath the skin of a patient 610. The cannula or needle 24 can have wings 26 around it for ease of placement of the needle or cannula and placement of the device at the target tissue site. The housing has opening 13 to attach the drug cartridge by passing it through opening 13 of the housing. The cartridge will be retained in position by friction with receiving column 17. However, force generated by the user's fingers can move the cartridge in position. It will be understood by those of ordinary skill in the art that after delivery of the first drug pellet, the cannula or needle can be re-positioned and another pellet can be delivered to the target area by moving the cartridge to align a second chamber for delivery. Thus, the device allows for sequential delivery of multiple pellets and one can triangulate these pellets around a pain generator or other target tissue site. For example, if a target tissue site generates pain, the physician can place multiple drug pellets containing an anti-inflammatory and/or analgesic agent around this pain generator.

In FIG. 4, the housing 11 is being affixed to coupling means 170 (e.g., threading, friction-fit fitting, luer connection, etc.) of the proximal end of the cannula 22, using wings 26 and grips 21 for easier assembly. Opening 13 of the housing is provided to slide the drug cartridge in between column 17 and the plunger is slid into housing opening 50. When disposed in the housing column 17, the drug cartridge will be able to be slid into the housing opening 13 and the user can align the drug pellet and the chamber so that the plunger can push the drug pellet out of the cartridge for delivery.

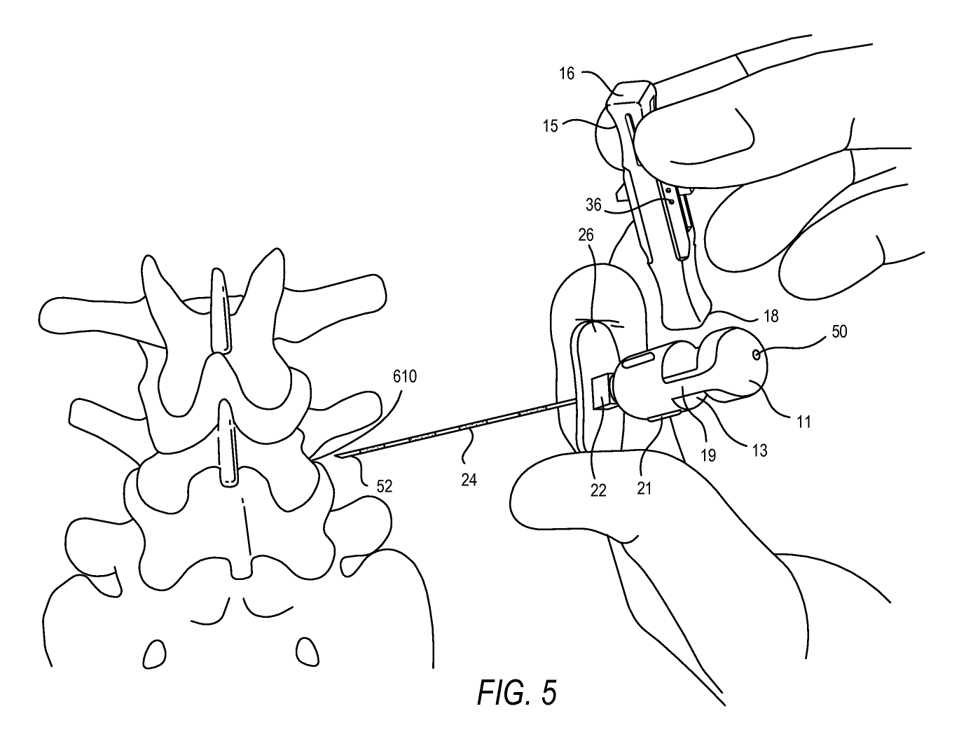

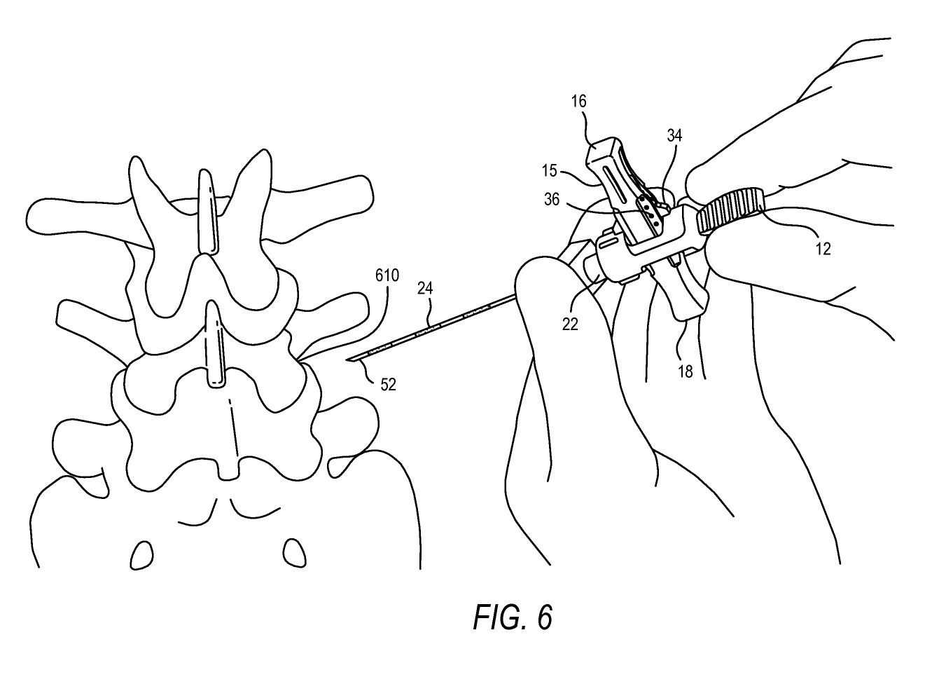

In FIG. 5, the cartridge 15 has distal end 18 that is being slid into the body of the housing 11 at opening 13, between columns (one column is shown in 19 and the other column is not shown). In use, the user grabs proximal end 16 to slide the drug cartridge. When disposed in the housing, the drug cartridge will be able to be slid into the housing to align the drug pellet and the chamber. Indicator 36 will let the user know that the pellet is aligned and in position for delivery. Shown in FIG. 6, the distal end 18 of the drug cartridge 15 is slid into the housing 11 and is aligned so that the drug pellet can be delivered. The plunger is shown in the extended position as the plunger handle contacts the surface of the housing 15. In some embodiments cover 34 can be pierced by the plunger.

In various embodiments, the drug depot comprises a drug cartridge containing drug pellets loaded within the chamber of the drug cartridge, when the plunger is moved to the extended position, the drug cartridge will remain within the housing and the chamber of the drug cartridge will guide the tip of the plunger longitudinally and the drug pellet will be released from it when it is in the extended position. A subsequent or second pellet may be administered by repositioning the needle at a target site, removing the plunger so that it is at a position above the drug cartridge, and sliding the drug cartridge at another position horizontal to the plunger and cannula to align the drug chamber and drug depot with the cannula and plunger. The plunger is then slid in a vertical direction within the housing to release the drug depot from the chamber into the cannula where the drug depot can be delivered to the target site by pushing it out the tip of the needle using the plunger. In this way, sequential delivery of a drug can be accomplished. Thus, the above procedure (e.g., repositioning the needle, removing the plunger, sliding the drug cartridge into position, inserting the plunger, delivering the drug depot) can be repeated multiple times to deliver multiple drug depots to the target tissue site.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A device for delivering a drug depot to a site beneath the skin of a patient, the device comprising:
a cannula having a proximal end and a distal end, the proximal end of the cannula having an opening to receive the drug depot, the distal end of the cannula capable of insertion to the site beneath the skin of the patient and having an opening for passage of the drug depot;
a drug cartridge comprising at least two chambers, wherein each chamber is configured to store and release a drug depot into the cannula when the cannula is aligned with a first chamber;
a housing having a top end, a bottom end, and a open portion, wherein the bottom end of the housing has a coupling means for coupling to the proximal end of the cannula and wherein the drug cartridge is slidably receivable within the open portion of the housing; and
a plunger having a handle and a tip adapted for expelling a first drug depot stored in a first chamber, wherein the tip of the plunger is slidably receivable within each of the housing, the first chamber of the cartridge, and the cannula to deliver the first drug depot to the site beneath the skin of the patient when at least the first chamber is aligned with the cannula and the plunger.

2. A device for delivering a drug depot according to claim 1, wherein the coupling means comprises threading disposed around the housing which is adapted to receive a threading disposed around the proximal end of the cannula.

3. A device for delivering a drug depot according to claim 2, wherein the threading on the bottom end of the housing and the proximal end of the cannula form a luer lock.

4. A device for delivering a drug depot according to claim 1, wherein the drug cartridge comprises an indicator to indicate alignment of the first chamber with the cannula and plunger for delivering the first drug depot to the site beneath the skin of the patient.

5. A device for delivering a drug depot according to claim 1, wherein the drug cartridge comprises an indicator to indicate alignment of the first chamber with the cannula and plunger for delivering the first drug depot to the site beneath the skin of the patient.

6. A device for delivering a drug depot according to claim 1, wherein each chamber of the drug cartridge has a proximal end and a distal end, the proximal end of the chamber having an opening to receive the drug depot and to receive the plunger when the chamber is uncovered, the distal end of the chamber having an opening for receiving the plunger and passage of the drug depot when the chamber is uncovered;
a cover slidably disposed on the proximal end and distal end of the at least one chamber; wherein movement of the cover so that the at least one chamber is uncovered allows movement of the plunger to an extended position, which allows movement of the drug depot out of the distal end of the chamber and out of the drug cartridge to deliver the drug depot to the site beneath the skin.

7. A device for delivering a drug depot according to claim 1, wherein the site is at least one muscle, ligament, tendon, cartilage synovial joint, bone, spinal disc, spinal canal, or a soft tissue surrounding the spinal canal.

8. A device for delivering a drug depot according to claim 1, wherein the first drug depot is contained in the first chamber and a second drug depot is contained in a second chamber of the drug cartridge and the first and the second drug depots are sterilizable and biodegradable drug pellets.

9. A device for delivering a drug depot according to claim 1, wherein the housing, drug cartridge, plunger, and/or cannula comprise a contoured surface for gripping the device.

10. A device for delivering a drug depot according to claim 1, wherein at least one of: (i) a handle is fixed to at least the cannula; (ii) the drug cartridge has indicator markings to indicate the number of drug depots delivered; or (iii) the plunger, cannula or drug depot include markings that indicate location at or near the site beneath the skin.

11. A kit for delivering a drug pellet to a site beneath the skin of a patient, the kit comprising: a sterilized drug delivery device, comprising:
a cannula having a proximal end and a distal end, the proximal end of the cannula having an opening to receive the drug pellet, the distal end of the cannula capable of insertion to the site beneath the skin of the patient and having an opening for passage of the drug pellet;
a drug cartridge comprising at least two chambers, wherein each chamber is configured to store and release the drug pellet into the cannula when the cannula is aligned with a first chamber of the drug cartridge;
a housing having a top end, a bottom end, and a open portion, wherein the bottom end of the housing has a coupling means for coupling to the proximal end of the cannula and the open portion is configured to receive the cartridge and wherein the drug cartridge is slidably receivable within the open portion of the housing; and
a plunger having a handle and a tip adapted for expelling a first drug pellet stored in the first chamber, wherein the tip of the plunger is slidably receivable within each of the housing, the first chamber of the cartridge, and the cannula to deliver the first drug pellet to the site beneath the skin of the patient when at least the first chamber is aligned with the cannula and the plunger.

12. A kit for delivering a drug pellet according to claim 11, wherein the drug delivery device can be assembled such that the housing is attached to the cannula; the cartridge is attached to the housing; and the plunger is slidably receivable through the housing, the cartridge, and the cannula, such that the drug pellet is expelled from the cartridge through the cannula to the site beneath the skin of the patient.

13. A kit for delivering a drug pellet according to claim 11, wherein each chamber of the drug cartridge has a proximal end and a distal end, the proximal end of the chamber having an opening to receive the drug pellet and to receive the plunger when the chamber is uncovered, the distal end of the chamber having an opening for receiving the plunger and passage of the drug pellet when the chamber is uncovered;
a cover slidably disposed on the proximal end and distal end of at least one chamber; wherein movement of the cover so that the at least one chamber is uncovered allows movement of the plunger to an extended position, which allows movement of the drug depot out of the distal end of the chamber and out of the drug cartridge to deliver the drug pellet to the site beneath the skin.

14. A method of delivering a drug pellet to a site beneath the skin of a patient, the method comprising:
inserting a cannula at the target tissue site, the cannula having a proximal end and a distal end, the proximal end of the cannula having an opening to receive a drug pellet, the distal end of the cannula capable of insertion to the site beneath the skin of the patient and having an opening for passage of the drug pellet;
attaching a drug cartridge to the proximal end of the cannula, the drug cartridge having a chamber containing the drug pellet; and
sliding the cartridge to align the drug pellet with the proximal end of the cannula and a plunger, the plunger having a handle and a tip adapted for expelling the drug pellet from the cannula, wherein the tip of the plunger is slidably receivable within the chamber and the cannula to deliver the drug pellet to the site beneath the skin of the patient.

15. A method according to claim 14, further comprising sliding the plunger within the chamber and the cannula to deliver the drug pellet to the site beneath the skin of the patient.

16. A method according to claim 14, wherein the attachment of the drug cartridge to the cannula is accomplished by first attaching a housing to the cannula, the housing configured to receive the drug cartridge and align the cannula with the chamber.

17. A method according to claim 14, wherein the drug cartridge comprises a second drug pellet disposed in a second chamber of the drug cartridge and the second drug pellet is delivered to the site beneath the skin by repositioning the cannula, removing the plunger from the cannula, sliding the cartridge to align the second drug pellet with the cannula, inserting the plunger and sliding the plunger within the second chamber of the drug cartridge and the cannula to deliver the second drug pellet to the site beneath the skin of the patient.

18. A method according to claim 17, wherein the drug cartridge is slid to the second chamber using index markings on the cartridge to ensure that the second chamber containing the second drug pellet is properly aligned with the cannula and the plunger tip.

19. A method according to claim 16, wherein the housing is attached to the cannula using a luer lock connection.

20. A method according to claim 14, wherein the site beneath the skin is at least one muscle, ligament, tendon, cartilage synovial joint, bone, spinal disc, spinal canal, or a soft tissue surrounding the spinal canal.
